(19) United States
(12) Patent Application Publication
(10) Pub. No.: US 2024/0268742 A1
(43) Pub. Date: Aug. 15, 2024

(54) IMPLANTABLE DEVICE

(71) Applicants: Murata Manufacturing Co., Ltd., Nagaokakyo-shi (JP); North Carolina State University, Raleigh, NC (US)

(72) Inventors: Shu HAMADA, Nagaokakyo-shi (JP); Alper BOZKURT, Morrisville, NC (US); James REYNOLDS, Graham, NC (US); Parvez AHMMED, Raleigh, NC (US)

(21) Appl. No.: 18/636,894

(22) Filed: Apr. 16, 2024

Related U.S. Application Data
(63) Continuation of application No. PCT/US2022/048201, filed on Oct. 28, 2022.
(60) Provisional application No. 63/273,369, filed on Oct. 29, 2021.

(51) Int. Cl. A61B 5/29; A61B 5/00; A61B 5/024

(52) U.S. Cl. CPC A61B 5/29; A61B 5/0031; A61B 5/02405

(57) ABSTRACT

An implantable device is provided that includes a tubular body with at least one open end, defining an inner space, a cap closing the at least one open end and having an inner surface on one side and an outer surface on the other side, the inner surface facing the inner space, and a circuit board being placed in the inner space, having an active surface, a board electrode disposed on the active surface, and a wireless communication circuit to communicate outside of a body, biological information obtained via the board electrode. Moreover, a recess and an inner electrode are disposed on the inner surface of the cap, and the circuit board is disposed in the recess, and the inner electrode is electrically connected to the board electrode. An outer electrode is disposed on the outer surface of the cap and is electrically connected to the inner electrode.

### CROSS REFERENCED TO RELATED APPLICATIONS

This application is a continuation of International Application No. PCT/US2022/048201, filed Oct. 28, 2022, which claims priority to U.S. Provisional Application No. 63/273,369, filed Oct. 29, 2021, the entire contents of each of which are hereby incorporated in their entirety.

### GOVERNMENT LICENSE RIGHTS

This invention was made with government support under grant number 1554367 awarded by the National Science Foundation. The government has certain rights in the invention.

### TECHNICAL FIELD

The present disclosure relates to implantable devices.

### BACKGROUND

Japanese publication JP 2021-511841A (hereinafter “JP '841”) describes, for example, an implantable device, including a body, a circuit board placed within the body, and an end-cap which closes the ends of the body.

The circuit board of the implantable device of JP '841 has a flexible distal portion. Before the end-cap is attached to the body, the flexible distal portion extends beyond the ends of the body. When the end-cap is attached, the flexible distal portion allows for the electrical connection between the circuit board and the end-cap.

However, regarding its assembly, the implantable device of JP '841 can be improved.

### SUMMARY

According to an exemplary aspect of the disclosure, provides an implantable device that can be assembled easily.

An implantable device according to an exemplary aspect of the present disclosure includes a tubular body with at least one open end, defining an inner space; a cap closing the at least one open end and having an inner surface on one side and an outer surface on the other side, the inner surface facing the inner space; and a circuit board being placed in the inner space, having an active surface, a board electrode disposed on the active surface, and a wireless communication circuit to communicate outside of a body, biological information obtained via the board electrode. Moreover, an inner electrode is disposed on the inner surface of the cap, and the circuit board is disposed in a recess. The inner electrode is electrically connected to the board electrode. In addition, an outer electrode is disposed on the outer surface of the cap and is electrically connected to the inner electrode.

An implantable device according to another exemplary aspect of the present disclosure includes a tubular body with at least one open end, defining an inner space; a cap closing the at least one open end and having an inner surface on one side and an outer surface on the other side, the inner surface facing the inner space; and a circuit board being placed in the inner space, having an active surface, a board electrode disposed on the active surface, and a wireless communication circuit to communicate outside of a body, biological information obtained via the board electrode. In the exemplary aspect, a protrusion is disposed on the inner surface of the cap and contacts the active surface, and an inner electrode is disposed on the protrusion and is electrically connected to the board electrode. In addition, an outer electrode is disposed on the outer surface of the cap and is electrically connected to the inner electrode. The device further includes a connection member connecting the inner electrode and the board electrode.

According to the present disclosure, an implantable device is provided that is easy to assemble.

Additional advantages and novel features of the system of the present disclosure will be set forth in part in the description that follows, and in part will become more apparent to those skilled in the art upon examination of the following or upon learning by practice of the disclosure.

### BRIEF DESCRIPTION OF THE DRAWINGS

In the descriptions that follow, like parts are marked throughout the specification and drawings with the same numerals, respectively. The drawings are not necessarily drawn to scale and certain drawings may be shown in exaggerated or generalized form in the interest of clarity and conciseness. The disclosure itself, however, as well as a mode of use, further features and advances thereof, will be understood by reference to the following detailed description of illustrative implementations of the disclosure when read in conjunction with reference to the accompanying drawings, wherein:

FIG. 1 is a schematic view of the implantable device in accordance with an exemplary aspect of the present disclosure;

FIG. 2 is a schematic view showing the inside of the implantable device;

FIG. 3 is a cross-sectional view of the implantable device;

FIG. 4 is a schematic view of the components forming the circuit of the circuit board;

FIG. 5 is a plane view of the cap;

FIG. 6 is an enlarged cross-sectional view of the cap and the circuit board;

FIG. 7 is a cross-sectional view of a first variation of the implantable device;

FIG. 8 is a cross-sectional view of a second variation of the implantable device;

FIG. 9A is a plane view of the cap;

FIG. 9B is an enlarged cross-sectional view of the cap and the circuit board;

FIG. 10 is a cross-sectional view of a third variation of the implantable device;

FIG. 11 is a cross-sectional view of a fourth variation of the implantable device;

FIG. 12 is a schematic view showing a practical example of the implantable device;

FIG. 13A is a figure showing raw ECG signals including respiratory artifacts as recorded by the implantable device;

FIG. 13B is a figure showing raw ECG signals including respiratory artifacts as recorded by the conventional ECG electrodes;

FIG. 14 is a figure showing filtered ECG signals after subtracting the respiratory artifacts;

FIG. 15A is a figure showing R-R intervals extracted using the clean ECG signals;

FIG. 15B is a figure showing respiratory intervals calculated from the gap between consecutive peaks in the ECG baseline;

FIG. 16A is a figure showing the magnitude of DFT applied on the R-R interval values from the implantable device; and

FIG. 16B is a figure showing the magnitude of DFT applied on the respiratory baseline from the implantable device.

### DETAILED DESCRIPTION

Continuous and wireless physiological monitoring would enable veterinarians, animal scientists, and biomedical researchers to better understand and monitor animal health and welfare. Traditional methods of animal health monitoring devices, especially for small animals, require benchtop instrumentation connected to the animals by wires and are only suitable for clinical settings. Moreover, external attachment of biopotential electrodes requires hair removal and adhesives, which make the animals uncomfortable with the devices during long-term use. A subcutaneously injected sensor, on the other hand, allows for free movement of animals and hence offers deeper insight into their physiological status in natural or semi-natural settings.

In general, electrocardiography (ECG) can provide some of the most important physiological endpoints that can be used to assess the animal's health and welfare status: heart rate (HR), heart rate variability (HRV), and potentially breathing rate (BR). In order to successfully integrate ECG recording circuitry into an implantable form, miniaturized electrodes are required. It is, however, challenging to manufacture such miniaturized electrodes. The electrodes need a consistent low-impedance connection from the body tissue to the circuit, while the electronics in the implant need to be completely insulated from the outside environment. This low-impedance connection requires the protrusion of conductors out of the implant packaging and a larger contact surface area between the electrode and the body tissue. Scalability and lower cost of the electrode manufacturing process are also necessary for commercial applications.

Existing electrode designs in the literature use either a spiraled wire or micromachined metal as the conductor. Some of the primary drawbacks of these methods include the requirement for manual production by hand, a difficult manufacturing process, increased cost, and the lack of uniformity of the end product.

Existing devices provide for a subcutaneous electrophysiology implantable device for monitoring HR using stainless steel wires as electrodes. With regards to this implantable device, the measurements were suboptimal due to the limited tissue-electrode contact and excessively small spacing between the electrodes. In other existing devices, electrodes made of conductive epoxy showed performance comparable to the other metallic alternatives. Electrodes made of conductive epoxy provided more flexibility in terms of implant packaging and encapsulation, but there is scope for improvement in terms of size, uniformity, and manufacturing complexity of the electrodes.

In order to advance the mass production of lower-cost biopotential electrodes, additive manufacturing (e.g., 3D-printing) technology can play a significant role. In the present disclosure, the possible adoption of a novel technique for printing metal electrodes on a co-fired ceramic base is described. By using this novel technology, caps can be manufactured for a tubular body (for example, a glass tube) with metal electrodes printed thereon. The metal electrodes can easily connect the external tissue surface to the electrophysiological front-end circuit within the implantable device.

By providing recesses or protrusions on caps with printed metal electrodes, it is easy to align the metal electrodes with the electrophysiological front-end circuit. Therefore, an implantable device can be provided that it easy to assemble.

An implantable device according to a first exemplary aspect includes a tubular body with at least one open end, defining an inner space; a cap closing the at least one open end and having an inner surface on one side and an outer surface on the other side, the inner surface facing the inner space; and a circuit board disposed in the inner space, having an active surface, a board electrode disposed on the active surface, and a wireless communication circuit configured to communicate outside of a body, biological information obtained via the board electrode. Moreover, an inner electrode is disposed on the inner surface of the cap and the circuit board is disposed in a recess. The inner electrode is electrically connected to the board electrode. An outer electrode is disposed on the outer surface of the cap and is electrically connected to the inner electrode.

According to this configuration, it is easy to align the circuit board in relation to the cap. Therefore, it is easy to assemble the implantable device.

The implantable device according to a second exemplary aspect, depending on the first aspect, further defines the recess by an inner wall having a first inner wall and a second inner wall facing each other.

According to this configuration, in the case where the first inner wall and the second inner wall face the circuit board, it is easy to hold the circuit board between the first inner wall and the second inner wall and keep the circuit board in an aligned position.

The implantable device according to a third exemplary aspect, depending on the second aspect, includes that the first inner wall is part of a first protrusion on the inner surface of the cap, the second inner wall is part of a second protrusion on the inner surface of the cap, and the recess is between the first protrusion and the second protrusion.

According to this configuration, regardless of the thickness of the cap and thus the depth of the recess, it is possible to hold the circuit board securely by the first protrusion and the second protrusion.

The implantable device according to a fourth exemplary aspect, depending on the third aspect, includes that at least one of the first protrusion and the second protrusion covers the circuit board on an end thereof.

The implantable device according to a fifth exemplary aspect, depending on any one of the second to fourth aspects, includes that the inner electrode is disposed on the inner wall, and the board electrode is disposed on a part of the circuit board which is disposed in the recess.

According to this configuration, electrical connection can be achieved between the inner electrodes and the board electrodes.

The implantable device according to a sixth exemplary aspect, depending on any one of the second to fifth aspects, includes that the circuit board contacts at least one of the first inner wall or the second inner wall.

According to this configuration, by increasing the contact area between the recess and the circuit board, the board can be held in a more secure manner.

The implantable device according to a seventh exemplary aspect, depending on the sixth aspect, includes that the circuit board contacts both the first inner wall and the second inner wall.

According to this configuration, by increasing the contact area between the recess and the circuit board, the board is held in a more secure manner.

The implantable device according to an eighth exemplary aspect, depending on any one of the first to seventh aspects, includes that the outer electrode has a larger surface area than the inner electrode.

The implantable device according to a ninth exemplary aspect, depending on any one of the first to eighth aspects, includes that an outer diameter of the cap is less than an inner diameter of the body.

According to this configuration, after assembling the aligned circuit board and the cap together, the circuit board and the cap can be inserted into the body.

The implantable device according to a tenth exemplary aspect, depending on any one of the first to ninth aspects, includes that the at least one open end is a first open end, the cap is a first cap, and the body has a second open end opposite the first open end, and the device further includes a second cap, which closes the second open end.

According to this configuration, the circuit board placed inside the body can be easily accessed from either end of the body.

The implantable device according to an eleventh exemplary aspect, depending on the tenth aspect, includes that an outer diameter of the first cap and an outer diameter of the second cap is less than an inner diameter of the body.

According to this configuration, both caps can be attached to the circuit board before inserting the circuit body into the body.

The implantable device according to a twelfth exemplary aspect, depending on any one of the first to eleventh aspects, includes that the cap includes a ceramic material and a metal material.

According to this configuration, the inner electrode and the outer electrode can be formed in an integrated manner with the cap.

The implantable device according to a thirteenth exemplary aspect, depending on any one of the first to twelfth aspects, includes that the cap includes a feed-through conductor penetrating the cap and electrically connecting the outer electrode and the inner electrode.

According to this configuration, electrical connection can be achieved between the inner electrode and the outer electrode.

The implantable device according to a fourteenth exemplary aspect, depending on the thirteenth aspect, includes that the feed-through conductor is tilted with regards to a thickness direction of the cap.

According to this configuration, a cap can be easily manufactured by a firing process.

An implantable device according to a fifteenth exemplary aspect includes a tubular body with at least one open end, defining an inner space; a cap closing the at least one open end and having an inner surface on one side and an outer surface on the other side, the inner surface facing the inner space; and a circuit board being placed in the inner space, having an active surface, a board electrode disposed on the active surface, and a wireless communication circuit to communicate outside of a body, biological information obtained via the board electrode. Moreover, a protrusion is disposed on the inner surface of the cap and contacts the active surface, an inner electrode is disposed on the protrusion and is electrically connected to the board electrode, and an outer electrode is disposed on the outer surface of the cap and is electrically connected to the inner electrode. The device further includes a connection member connecting the inner electrode and the board electrode.

According to this configuration, it is easy to align the circuit board in relation to the cap. Therefore, it is easy to assemble the implantable device.

The implantable device according to a sixteenth exemplary aspect, depending on the fifteenth aspect, includes that the inner electrode is disposed on the protrusion, on the other side to the side contacting the active surface, and the connection member is electrically conductive.

According to this configuration, compared to the case where the inner electrode is formed on the surface which contacts the active surface, it is easy to provide the connection member.

The implantable device according to a seventeenth exemplary aspect, depending on the fifteenth or sixteenth aspect, includes that the cap has an outer diameter that is less than an inner diameter of the tubular body.

The implantable device according to an eighteenth exemplary aspect, depending on any one of the fifteenth to seventeenth aspects, includes that the at least one open end is a first open end, the cap is a first cap, and the tubular body has a second open end that is opposite the first open end, wherein the implantable device further comprises a second cap that closes the second open end, and wherein the first cap and the second cap each have an outer diameter that is less than an inner diameter of the tubular body.

The implantable device according to a nineteenth exemplary aspect, depending on any one of the fifteenth to eighteenth aspects, includes that the cap comprises a feed-through conductor that penetrates the cap and that electrically connects the outer electrode and the inner electrode, and wherein the feed-through conductor is tilted with regards to a thickness direction of the cap.

The implantable device according to a twentieth exemplary aspect, depending on any one of the fifteenth to nineteenth aspects, includes that the protrusion is a first protrusion, wherein the implantable device further comprises a second protrusion disposed on the inner surface of the cap, wherein the circuit board is disposed between the first protrusion and the second protrusion.

Below, an exemplary aspect of the disclosure is described with reference to the figures. It should be appreciated that in each figure below, each component has been exaggerated for the sake of clarity.

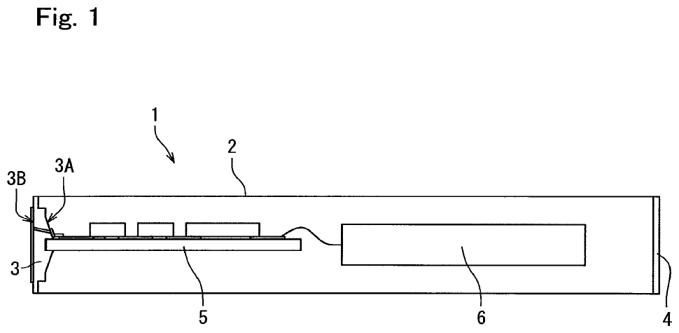

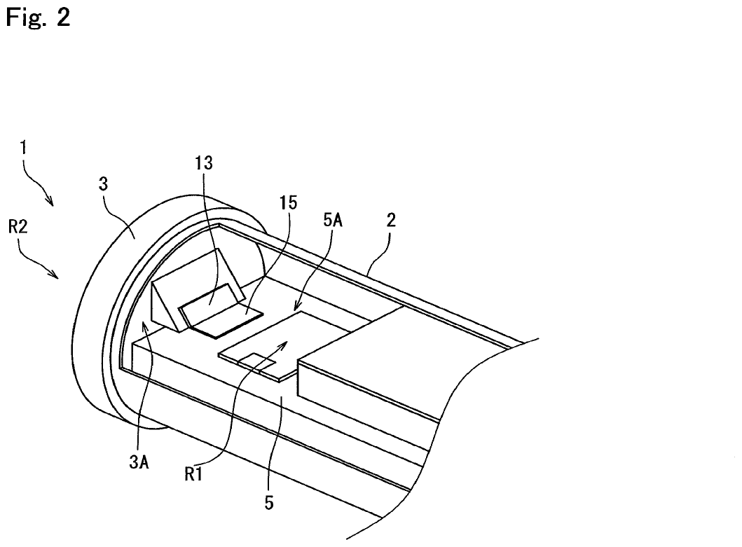

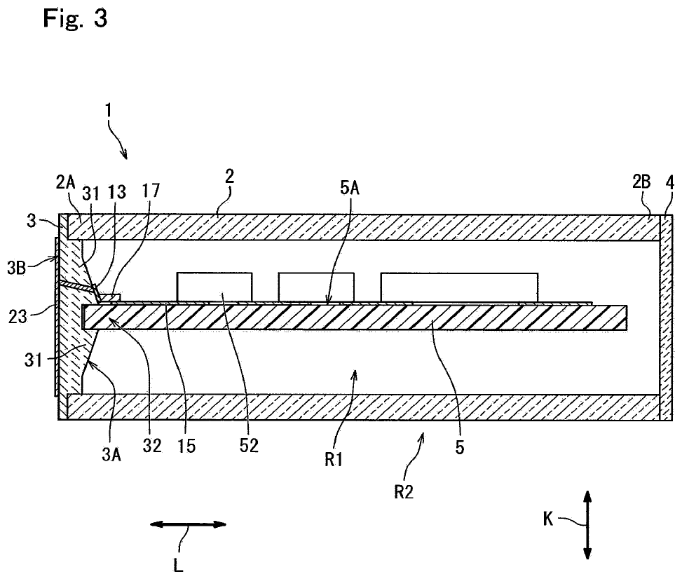

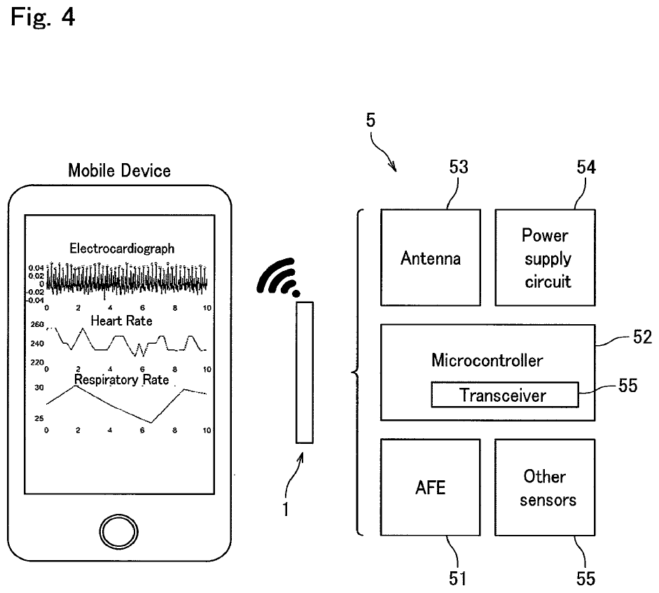

FIG. 1 is a schematic view of the implantable device 1 in accordance with an exemplary aspect of the disclosure. FIG. 2 is a schematic view showing the inside of the implantable device 1. FIG. 3 is a cross-sectional view of the implantable device 1. FIG. 4 is a schematic view of the components forming the circuit of the circuit board 5.

For example, the implantable device 1 is a device implanted into animals in order to obtain biological information. For example, the implantable device 1 can be implanted into chickens and the biological information includes an electrocardiogram.

As shown in FIG. 1 to FIG. 3, the implantable device 1 includes a body 2, two caps 3, 4, and a circuit board 5. The circuit board 5 is housed in the body 2, and both ends of the body 2 are closed by caps 3, 4.

FIG. 1 shows an example in which the implantable device 1 further includes a battery 6 as the electrical power supply. In this case, the battery 6 is aligned with the circuit board 5 and placed between the circuit board 5 and the cap 4. The present disclosure is not, however, limited to this configuration and other forms of electrical power supply are possible. In the other figures, the battery 6 is omitted for simplicity.

The body 2 is a tubular body that houses the circuit board 5. The body 2 defines an inner space R1 within, and the circuit board 5 is placed inside the inner space R1 in its entirety. For purposes of this disclosure, the inner space R1 means the space inside the body 2, while, in contrast, the outer space R2 means the space outside the body 2, i.e., the space contacting body tissue. In one aspect of the disclosure, the body 2 is a cylindrical member with two open ends opposite each other, and the outer diameter of the end of the body 2 is smaller than the length of the body 2. The inner diameter of the body 2 is, for example, 4 mm.

Here, the direction passing through the center of both ends of the body 2 is defined as the axial direction L. The axial direction L of the body 2 is parallel to the thickness direction of the cap 3 and is parallel to the longitudinal direction of the circuit board 5.

The body 2 may be made of glass. In particular, the body 2, for example, is made of borosilicate glass in an exemplary aspect.

As shown in FIG. 3, the caps 3, 4 are circular disc-shaped members which respectively close the open ends 2A, 2B of the body 2. By closing the body 2 with the caps 3, 4, the inner space R1 is sealed and the inner space R1 and the outer space R2 are separated.

The cap 3 is electrically connected to one end 2A of the circuit board 5. The cap 4 may be separated from the other end 2B of the circuit board 5.

In order to achieve an electrical connection with the circuit board 5, the cap 3 has electrodes. Firstly, the cap 3 has an inner surface 3A on one side, and an outer surface 3B on the other side. The inner surface 3A faces the inner space R1 and the outer surface 3B faces the outer space R2. An inner electrode 13 is disposed on the inner surface 3A and an outer electrode is disposed on the outer surface 3B. The inner electrode 13 is electrically connected to the circuit board 5, and the outer electrode 23 is electrically connected to the body tissue in the outer space R2. The outer electrode 23 and the inner electrode 13 are electrically connected to each other by a feed-through conductor 18 which penetrates the cap 3. Therefore, a biopotential generated in the outer space R2 is conducted to the circuit board 5 via the outer electrode 23 and the inner electrode 13.

In one aspect of the disclosure, the cap 3 has one inner electrode 13 and one outer electrode 23. The outer electrode 23 is larger in surface area than the inner electrode 13. Thus, the outer electrode 23 ensures a large contact area with the body tissue.

The inner surface 3A of the cap 3 has two protrusions 31. The protrusions 31 protrude from the inner surface 3A, along the circuit board 5 in the axial direction L. A recess 32 is formed on the inner surface 3A of the cap 3 between the two protrusions 31. The circuit board 5 is disposed in the recess 32. The recess 32 is recessed in regard to the tip of the protrusions 31, and creates an inner space between the protrusions 31. When the circuit board 5 is inserted into the recess 32, the two protrusions 31 are configured to “sandwich” the circuit board 5 in the thickness direction K. According to this configuration, it is easy to align the circuit board 5 in regard to the cap 3 by guiding the circuit board 5 with the protrusions 31 and inserting the circuit board 5 into the recess 32.

For purposes of this disclosure, a “protrusion” means a configuration that is protruding compared to its surroundings, and a “recess” means a configuration that is recessed compared to its surroundings.

According to an exe4mplary aspect, the inner electrode 13 is positioned on one of the protrusions 31. The protrusion 31 extends along the circuit board 5, so that by providing the inner electrode 13 on the protrusion 31, it is possible to position the inner electrode 13 close to the electrode on the circuit board 5 (i.e., the board electrode 15 as later described), even when the electrode on the circuit board 5 is positioned inwardly with respect to the end of the circuit board 5.

The circuit board 5 is equipped with an electronic circuit configured to obtain biological information related to the outer space R2, and further to communicate (e.g., transmit) the biological information to a device or a system outside the body. The electronic circuit is configured with a circuit pattern formed on the circuit board 5 and electronic components electrically connected to the circuit pattern. For example, the circuit board 5 is a printed circuit board. As shown in FIG. 2, the circuit board 5 is plate-shaped and at least one of the surfaces of the circuit board 5 is an active surface 5A onto which the electronic components are mounted.

As shown in FIG. 3, the circuit pattern and a board electrode 15 are disposed on the active surface 5A of the circuit board 5, and multiple electronic components are mounted onto the active surface 5A.

The board electrode 15 is disposed near the end of the circuit board 5. In one aspect of the disclosure, the board electrode 15 is disposed inwardly with respect to from the end of the circuit board 5, so that a gap is formed between the board electrode 15 and the inner electrode 13 in the axial direction L. By positioning the board electrode 15 away from the end of the circuit board 5, when machining the end of the circuit board 5, it is possible to avoid machining the conducting wires and to suppress burr generation.

The board electrode 15 is connected to the inner electrodes 13 via a connection member 17. The connection member 17 is electrically conductive and electrically connects the inner electrode 13 and the circuit board 5. The connection member 17 is made of a conductive material. For example, the connection member 17 is solder or a conductive adhesive. The connection member 17 also fixes the circuit board 5 to the inner electrode 13. In other words, the connection member 17 is a member for electrically and mechanically connecting the inner electrode 13 and the circuit board 5.

The circuit board 5 has mounted electronic components which are electrically connected to the inner electrode 13 of the cap 3 via the board electrode 15 and the connection member 17. The mounted electronic components include at least a control circuit, a wireless communication circuit, a power supply circuit and an analog-digital (AD) conversion circuit. The control circuit is a controller which controls the entire implantable device 1. In an exemplary aspect, the control circuit includes a general-purpose processor, such as CPUs, MPUs, FPGAs, DSPs, and ASICs, that is configured to perform predetermined functions by executing programs. The control circuit can achieve various controls in the implant device 1 by calling and executing control programs stored in a memory (not shown). The control circuit is not limited to those that achieve a given function through the cooperation of hardware and software, but may also be a hardware circuit designed specifically for achieving a given function. The wireless communication circuit communicates biological information outside of the body. The AD conversion circuit converts analog signals into digital signals.

As shown in FIG. 4, in one aspect of the disclosure, the mounted electronic components include an analog front-end 51 with an AD conversion circuit, a microcontroller 52 with a control circuit, an antenna 53 with a wireless communication circuit, and a power supply circuit 54. According to this configuration, under the control of the microcontroller 52, the circuit board 5 communicates biological information measured by the analog front-end to external devices or systems.

The analog front-end 51 (for example, MAX30003, 2.9×2.7×0.6 mm³, Maxim Integrated, San Jose, CA, USA) is configured to measure the biological information in the outer space R2. In one aspect of the disclosure, the analog front-end 51 measures heart rate based on an electrocardiogram (ECG). The analog front-end 51 has an analog-digital converter to convert the measured analog signals into digital signals. The analog front-end 51 may be connected to the board electrode 15 via a network of components which act as a signal filter.

The microcontroller 52 controls the implantable device 1. The microcontroller 52 is combined with a radio transceiver 55 (for example, CC2640, 2.7×2.7×0.5 mm³, Texas Instruments, Dallas, TX, USA) to form a system-on-a-chip. The radio transceiver 55 acts as a wireless communication circuit, and has a BLE (Bluetooth® Low Energy) radio for said wireless communication. The radio transceiver 55 may perform wireless communications and transmit and/or receive data according to other standards.

The radio transceiver 55 communicates via an antenna 53. The antenna 53 (for example, 2450AT, 5.0×2.0×1.5 mm³, Johanson Technology, Inc., Camarillo, CA, USA) is an integrated RF antenna and for example, communicates via 2.4 GHz radio waves.

The power supply circuit 54 is a circuit connected to a power supply (not shown) in order to supply the circuit board 5 with power. In one aspect of the disclosure, the power supply is a pin-type Li-ion battery with a diameter of 3.6 mm (see FIG. 1), which is attached to one end of the circuit board 5 via a wire and solder.

In addition, it is noted that other electronic components may be mounted on the circuit board 5. For example, a memory for storing the biological information may be mounted on the circuit board 5. The memory is a recording medium which records various information and control programs, and may be a memory that functions as a working area for the microcontroller 52. The memory may be, for example, flash memory, SSD (Solid State Device), hard disk, RAM, or other storage devices, or a combination thereof, as appropriate.

Further, electronic components which obtain biological information other than electrocardiograms may be mounted on the circuit board 5. For example, a thermistor for measuring body temperature, or an accelerometer for measuring movement may be mounted on the circuit board 5.

For example, each electronic component is soldered onto the circuit board 5. After the electronic components are soldered onto the circuit board 5, the circuit board 5 and the electronic components may be coated with a biomedical epoxy, and the circuit board 5 and the electronic components may be sealed.

The width of the circuit board 5 is smaller than the inner diameter of the body 2 so that it can be accommodated within the inner space R1. For example, the width of the circuit board 5 is 3.6 mm. The circuit board 5 may be positioned in contact with the body 2. Below, the cap 3 will be described in detail.

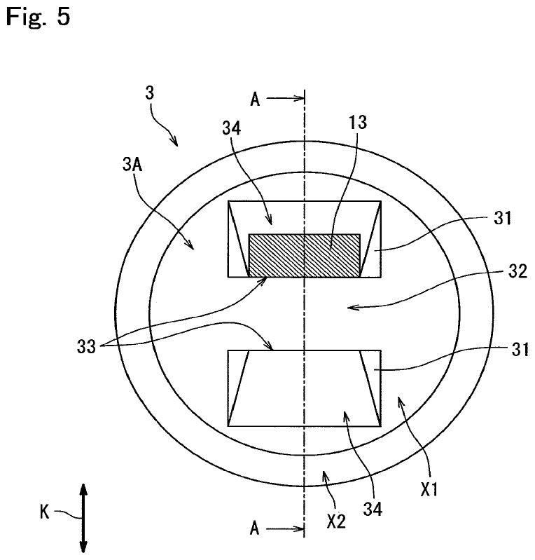

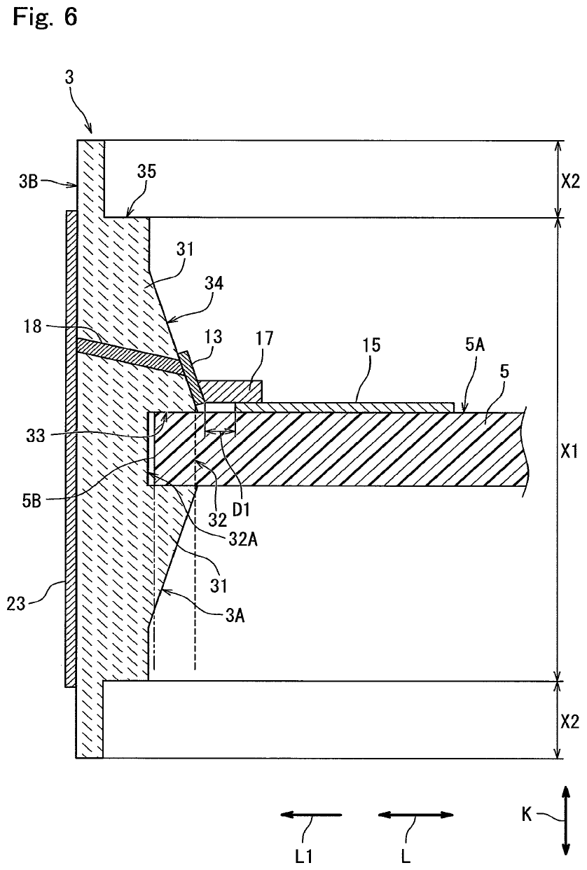

FIG. 5 is a plane view of the cap 3, showing the inner surface 3A. In FIG. 5, hatching has been applied to the inner electrodes 13 for clarity. FIG. 6 is an enlarged cross-sectional view of the cap 3 and the circuit board 5. The cross-section shown in FIG. 6 is the cross-section along the line AA in FIG. 5.

As shown in FIG. 5, each of the protrusions 31 has a contact surface 33 on one side and a non-contact surface 34 on the other side. The one side of the tip of the protrusion 31 is the contact surface 33 and the other side is the non-contact surface 34. The contact surfaces 33 of the two protrusions 31 are placed on either side of the recess 32 opposing each other. Herein, surfaces opposing each other may be taken to mean that the surfaces are facing each other and are substantially parallel to each other. The spacing between the contact surfaces 33, thus the dimension of the recess 32 in the thickness direction K may be roughly 1 mm.

As shown in FIG. 6, the circuit board 5 contacts both of the contact surfaces 33 and one of the contact surfaces 33 contacts the active surface 5A. The non-contact surfaces 34 face away from the circuit board 5. The inner electrode 13 is disposed the protrusion 31 contacting the active surface 5A, on the non-contact surface 34. In one aspect of the disclosure, the inner electrode 13 is disposed on the non-contact surface 34 near the tip of the protrusion 31.

When the cap 3 is made of an elastic material, such as a plastic, the inner electrode 13 may be disposed on the contact surface 33. If the cap 3 is made of an elastic material, it is possible to obtain a stable electrical connection just by inserting the circuit board 5 into the recess 32. Thus, in this case, the connection member 17 can be omitted from the configuration of the implantable device 1.

As shown in FIG. 5, the width of the protrusion 31 becomes smaller the further it extends away from the inner surface 3A. According to this configuration, it is possible to secure more space on the circuit board 5 for the electronic components.

As shown in FIG. 6, the contact surfaces 33 extend along the axial direction L. In one aspect of the disclosure, the contact surfaces 33 are parallel to the active surface 5A of the circuit board 5. According to this configuration, the circuit board 5 can be held in place in a stable manner using the contact surfaces 33, and at the same time, to place the inner electrodes 13 closer to the board electrodes 15. On the other hand, the non-contact surfaces 34 are tilted with regards to the active surface 5A so that the non-contact surface 34 approaches the contact surface 33 the further it extends along the circuit board 5.

The inner surface 3A can be divided into a first region X1 placed in the inner space R1 and a second region X2 which surrounds the first region X1. The second region X2 faces the end 2A of the body 2 (see FIG. 3). The outer diameter of the first region X1 is approximately the same as the inner diameter of the body 2. For example, the outer diameter of the first region X1 is sufficiently small so that it can be inserted into the inner space R1, while it is sufficiently large enough so that it can be secured inside the inner space R1. The outer diameter of the second region X2 (thus, the outer diameter of the cap 3) is larger than the inner diameter of the body 2 and is, for example 5 mm. The thickness of the cap 3 in the second region X2 is smaller than the thickness of the cap 3 in the first region X1, so that in between the second region X2 and the first region X1, a wall 35 extends in the axial direction L. By contacting the wall 35 to the inner surface of the body 2, it is possible to prevent the entry of liquids from the outer space R2 into the inner space R1. Furthermore, it is easy to align the centers of the cap 3 and the body 2.

For purposes of this disclosure, the outer diameter of a given component means the maximum distance between two points on the outer circumference of a cross-section of the component. Moreover, the inner diameter of a given component means the maximum distance between two points on the inner circumference of a cross-section of the component.

The feed-through conductor 18, which electrically connects the inner electrode 13 and the outer electrode 23, is, for example made of the same material as the inner electrode 13 and/or the outer electrode 23. The feed-through conductor 18 is tilted with regards to the axial direction L. Specifically, the feed-through conductor 18 is tilted so that it approaches the center of the cap 3 as it progresses from the outer surface 3B to the inner surface 3A. According to this configuration, cracks in the feed-through conductor 18 can be prevented and the cap can be easily manufactured using a firing process.

In one aspect of the disclosure, the base material of the cap 3 is made of a ceramic material, while the inner electrode 13, the outer electrode 23, and the feed-through conductor are made of copper or any other suitable metal material. The cap 3 is manufactured by a method that combines a multi-material 3D printing process with custom inks for ceramic, copper, and support materials. A different custom ink may be used for each of the materials.

Firstly, the cap 3 is designed using a commercial CAD tool (for example, 3D-CAD, Autodesk, Inc., San Rafael, CA, USA). The files which contain the design of the cap 3 are converted so that they can be used in an inkjet printer. The inkjet printer heads simultaneously layer several inks, i.e., an ink containing low-temperature co-fired ceramic (LTCC), an ink containing copper, and an ink containing a support material, on a heated platform. These layers harden as the solvents evaporate. Further, as the melting and sintering speed for ceramics and copper are different, the feed-through conductor 18 is formed in a tilted manner with regards to the axial direction L. After the layering of materials is complete, the resulting layered structure is placed in a hypoxic environment containing N₂/H₂ atmosphere and heated to above 800° C. This co-firing process removes the support material, resulting in the formation of cap 3. Finally, the surface of the electrodes 13, 23 are coated with electroless nickel immersion gold (ENIG).

According to the above method, it is possible to manufacture a cap 3 with connected electrodes 13, 23 as shown in FIG. 6.

Below, the relationship between the position of cap 3 and the position of circuit board 5 is explained in detail.

As shown in FIG. 6, the protrusion 31 of cap 3 covers the circuit board 5 near the end 5B of the circuit board 5. The inner electrode 13 is disposed near the tip of the protrusion 31 which covers the active surface 5A of the circuit board 5. Therefore, the end 5B of the circuit board 5 is positioned more externally L1, i.e., closer to the outer surface 3B compared to the inner electrode 13. This allows the inner electrode 13 to be placed closer to the board electrode 15.

Additionally, in one aspect of the disclosure, the bottom 32A of the recess 32 is positioned more externally L1 compared to the inner electrode 13. The end 5B of the circuit board 5 is positioned close to the bottom 32A of the recess 32.

According to such positional relationships, the distance D1 in the axial direction L between the inner electrode 13 and the board electrode 15, which is disposed inwardly with regards to the end 5B of the circuit board 5, can be made smaller. Therefore, along with making the implantable device smaller, the connection member 17 connecting the inner electrode 13 and the board electrode 15 can be made shorter, thus reducing the noise in the measurement results produced by circuit board 5.

Here, the assembly method of the implantable device is explained. Firstly, a part of the circuit board 5 is inserted into the recess 32 of the cap 5. By inserting the circuit board 5 into the recess 32, it is easy to align the circuit board 5 with regards to the cap 3. Next, while the circuit board 5 is in an aligned state, the circuit board 5 and the cap 3 are secured to each other by the connection member 17. Specifically, the circuit board 5 and the cap 3 are soldered together. By soldering, the circuit board 5 and the cap 3 can be connected electrically and mechanically while maintaining their correct positional relationship. As a result, the circuit board 5 and the cap 3 conduct electricity and are secured to each other. The correct positional relationship is a relationship which allows for the electrical connection between the inner electrode 13 and the board electrode 15, and prevents interference between the electronic components of the circuit board 5 and the inner surface of the body 2. Next, the circuit board 5, which has been connected to the cap 3, is inserted into the body 2, and thus the cap 3 closes one end 2A of the body 2. Next, the cap 4 closes the other end 2B of the body 2. The two caps 3, 4 can be attached to the body 2 by any suitable means and/or components for attaching two parts together. Below, an exemplary usage of the implantable device 1 will be described.

The implantable device 1 can be implanted into the body of an animal (e.g., a chicken) using local anesthesia and an external (i.e., ex vivo, outside of the body in which the implantable device 1 is implanted) computer can be used to collect biological information.

Specifically, the analog front-end 51 obtains biological information, such as electrocardiograms related to the outer space R2, via the board electrodes 15, the outer electrodes 23 and the inner electrodes 13. The analog front-end 51 converts biopotential signals into digital signals and transmits the digital signals to the microcontroller 52. The analog front-end 51 transmits the digital signals to the microcontroller 52, for example, via a SPI bus. When the external computer establishes a connection with the implantable device 1, the microcontroller 52 transmits the digital signals to the external computer in real-time using the radio transceiver 55. According to this configuration, it is possible to obtain the biological information in real-time.

According to the implantable device 1 in one aspect of the disclosure, the following effects can be achieved.

The implantable device 1 of one aspect of the disclosure includes a tubular body 2 with at least one open end 2A, defining an inner space R1. The implantable device 1 also includes a cap 3 closing the at least one open end 2A and having an inner surface 3A on one side and an outer surface 3B on the other side, the inner surface 3A facing the inner space R1. The implantable device 1 also includes a circuit board 5 being placed in the inner space R1, having an active surface 5A, a board electrode 15 disposed on the active surface 5A, and an antenna 53 (e.g., a wireless communication circuit) to communicate outside of a body, biological information obtained via the board electrode 15. A recess 32 is formed and an inner electrode 13 is disposed on the inner surface 3A of the cap 3, and the circuit board 5 is inserted in the recess 32, and the inner electrode 13 is electrically connected to the board electrode 15. An outer electrode 23 is disposed on the outer surface 3B of the cap 3, the outer electrode 23 is electrically connected to the inner electrode 13.

According to this configuration, by providing a recess 23 on the cap 3, it is easy to align the circuit board 5 to the cap 3 and it is also easy to hold circuit board 5 in place. Thus, it is easy to assemble implantable device 1.

Regarding the implantable device 1 of one aspect of the disclosure, the recess 32 is defined by an inner wall having a contact surface 33 (e.g., a first inner wall) and another contact surface 33 (e.g., a second inner wall) facing each other.

According to this configuration, it is possible to “sandwich” the circuit board 5 in between opposing contact surfaces 33 so that the circuit board 5 is held securely in place.

Regarding the implantable device 1 of one aspect of the disclosure, the contact surface 33 is part of a first protrusion 31 on the inner surface 3A of the cap 3, and the other contact surface 33 is part of a second protrusion 31 on the inner surface 3A of the cap 3. The recess 32 is between the first protrusion 31 and the second protrusion 31.

According to this configuration, regardless of the thickness of the cap 3 and thus the depth of the recess 32, it is possible to make protrusions 31 with a large enough protrusion to securely hold the circuit board 5 in place.

Regarding the implantable device 1 of one aspect of the disclosure, the circuit board 5 contacts at least one of the contact surfaces 33.

According to this configuration, by increasing the surface area of contact between the recess 32 and the circuit board 5, it is possible to securely hold the circuit board 5 in place.

Regarding the implantable device 1 of one aspect of the disclosure, the circuit board 5 contacts both contact surfaces 33.

According to this configuration, by increasing the surface area of contact between the recess 32 and the circuit board 5, the circuit board 5 can be securely held in place.

The implantable device 1 of one aspect of the disclosure includes a tubular body 2 with at least one open end 2A, defining an inner space R1. The implantable device 1 also includes a cap 3 closing the at least one open end 2A and having an inner surface 3A on one side and an outer surface 3B on the other side, the inner surface 3A facing the inner space R1. The implantable device 1 also includes a circuit board 5 being placed in the inner space R1, having an active surface 5A, a board electrode 15 disposed on the active surface 5A, and an antenna 53 to communicate outside of a body, biological information obtained via the board electrode 15. A protrusion 31 is disposed on the inner surface 3A of the cap 3 and contacts the active surface 5A. An inner electrode 13 is disposed on the protrusion 31 and is electrically connected to the board electrode 15. An outer electrode 23 is formed on the outer surface 3B of the cap 3 and is electrically connected to the inner electrode 13. The implantable device 1 further includes a connection member 17 connecting the inner electrode 13 and the board electrode 15.

According to this configuration, it is easy to align the circuit board 5 to the cap 3 and thus it is easy to assemble the implantable device 1. In addition, by providing the protrusion 31, the inner electrode 13 can be easily positioned closer to the board electrode 15. Therefore, it is possible to achieve the miniaturization of the implantable device 1, as well as making the distance between the electrodes shorter to reduce noise.

Regarding the implantable device 1 of one aspect of the disclosure, the inner electrode 13 is disposed on the protrusion 31, on the other side to the side contacting the active surface 5A, and the connection member 17 is electrically conductive.

According to this configuration, it is possible to keep a stable electrical connection between the inner electrode 13 and the board electrode 15 via the connection member 17. It is also easier to provide the connection member 17, compared to a configuration where the inner electrode 13 is disposed on the contact surface 33.

Regarding the implantable device 1 of one aspect of the disclosure, the at least one open end 2A is a first open end, the cap 3 is a first cap, and the tubular body 2 has a second open end 2B opposite the first open end 2A, and the implantable device 1 further includes a second cap 4, which closes the second open 2B.

According to this configuration, it is possible to have both ends 2A, 2B of the body 2 open at the start of the manufacturing process for the implantable device 1, and then have both ends 2A, 2B closed by the end of the manufacturing process. This may enable access to the circuit board 5 from both ends 2A, 2B of the body 2 during the manufacturing process, which may facilitate the manufacture of the implantable device 1.

Regarding the implantable device 1 of one aspect of the disclosure, the cap 3 includes a ceramic material and a metal material.

According to this configuration, the inner electrode 13 and the outer electrode 23 can be integrated into the cap 3. Therefore, the structure of the implantable device 1 becomes simpler and its assembly easier.

Regarding the implantable device 1 of one aspect of the disclosure, the cap 3 includes a feed-through conductor 18 penetrating the cap 3 and electrically connecting the outer electrode 23 and the inner electrode 13.

According to this configuration, an electrical connection between the inner electrode 13 and the outer electrode 23 can be achieved.

Regarding the implantable device 1 of one aspect of the disclosure, the feed-through conductor 18 is tilted with regards to an axial direction L (e.g., a thickness direction of the cap 3).

According to this configuration, the cap 3 can be easily manufactured by firing. In addition, it is possible to prevent the generation of cracks in the feed-through conductor 18.

In one aspect of the disclosure, for example, in which both ends 2A, 2B of the body 2 are open, is described, but the disclosure is not limited to this configuration. The body 2 is a tubular body with at least one open end. For example, one end of the body 2 may be closed and the cap 4 may be omitted.

In one aspect of the disclosure, for example, in which the body 2 is cylindrical, is described, but the disclosure is not limited to this configuration. For example, the body 2 may be a tubular member with a polygon-shaped cross-section. In this case, the shape of caps 3, 4 may be designed in accordance with the cross-section of the body 2.

In one aspect of the disclosure, for example, in which the protrusions 31 “sandwich” the circuit board 5 from the thickness direction K, is described, but the disclosure is not limited to this configuration. For example, the protrusions 31 may be arranged to “sandwich” the circuit board 5 from a width direction. Additionally, the protrusions 31 may be arranged to “sandwich” the circuit board 5 from both the thickness direction K and the width direction. In this case, the protrusions 31 may surround the circuit board 5 and the protrusions 31 may be formed as one continuous protrusion.

In one aspect of the disclosure, for example, in which the contact surface 33 contacts the circuit board 5 and is parallel to the active surface 5A of the circuit board 5, is described, but the disclosure is not limited to this configuration. For example, the contact surface 33 may have a curved shape with regards to the circuit board 5 and another material, for example an adhesive or a filler, may be placed between the contact surface 33 and the circuit board 5.

In one aspect of the disclosure, for example, in which the non-contact surface 34 of the protrusions 31 are tilted and the protrusion 31 has a triangular-shaped cross-section, is described, but the disclosure is not limited to this configuration. The protrusion 31 may have a differently shaped cross-section, for example a rectangular cross-section along the axial direction L. On the other hand, the alignment of the circuit board 5 to the cap 3 is facilitated if the protrusion 31 is shaped so that the contact surface 33 extends along the circuit board 5.

In one aspect of the disclosure, for example, in which the microcontroller 52 transmits digital signals to the external computer, is described, but the disclosure is not limited to this configuration. In place of a computer, an alternative external data aggregator may be provided to establish communication with the implantable device 1 via BLE.

In one aspect of the disclosure, for example, in which the cap 3 has two protrusions 31, is described, but the disclosure is not limited to this configuration. For example, as show in the first variation, the cap 3 may be provided with only one protrusion 31, which contacts the active surface 5A of the circuit board 5.

In addition, as shown in the second variation, the cap 3 may not have protrusions 31.

In one aspect of the disclosure, for example, in which the implantable device 1 has caps 3, 4, is described, but the disclosure is not limited to this configuration. For example, the implantable device 1 may have two caps 3. In this case, for example, as shown in the third variation, the outer diameter of the first cap 3 and the outer diameter of the second cap 3 may be less than the inner diameter of the body 2, easily enabling the assembly of the implantable device 1.

In one aspect of the disclosure, for example, in which one of the two protrusions have the inner electrode 13, is described, but the disclosure is not limited to this configuration. For example, as shown in the fourth variation, the cap 3 may be provided with inner electrodes 13 on both protrusions 31, the inner electrodes facing the circuit board 5. In the case where both surfaces of the circuit board 5 are active surfaces, a cap 3 having inner electrodes on both of the two protrusions 31 would improve the design freedom of the circuit board 5.

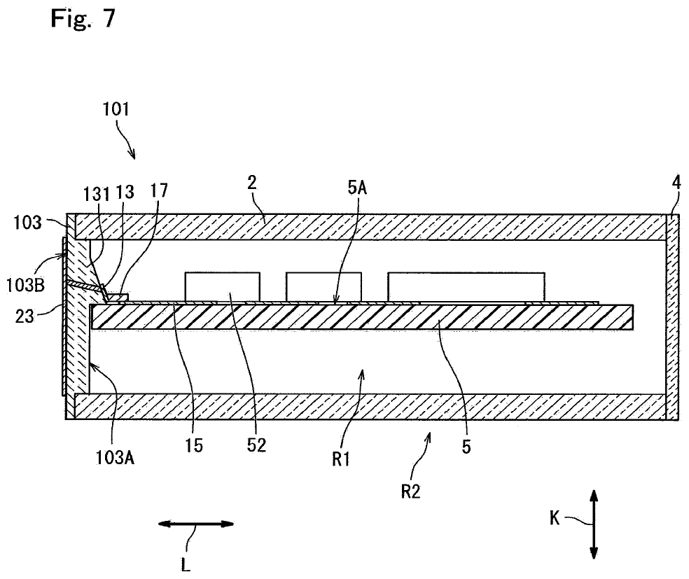

FIG. 7 is a cross-sectional view of the implantable device 101 of the first variation of the exemplary aspect. As shown in FIG. 7, the implantable device 101 of the first variation differs from the implantable device 1 of the above-described aspect in that it has a cap 103. It is noted that with regards to the first variation, unless stated otherwise, the other structures of implantable device 101 are the same as those of the implantable device 1 of one aspect of the disclosure, and the structures which are the same or equivalent to those of one aspect of the disclosure, will be referred to and explained using the same reference numbers. The same applies to the other variations that follow.

In particular, a protrusion 131 is disposed on the inner surface 103A of the cap 103. The protrusion 131 protrudes in the axial direction L of the body 2 and contacts the active surface 5A of the circuit board 5. In other words, the protrusion 131 acts as a structure to perform the alignment of the circuit board 5 in the thickness direction K within the body 2. The circuit board 5 is mechanically connected to the protrusion 131 by the connection member 17 to prevent the circuit board 5 from moving away from the protrusion 131. By providing the protrusion 131, the circuit board 5 can be prevented from coming into close proximity with the inner surface of the body 2, and thus preventing the electronic components on the circuit body 5 from hitting the inner surface of the body 2, using a simple structure. Therefore, the circuit board 5 can be prevented from being damaged. The inner surface 103A of the cap 103 may also have a recess 32.

The inner electrodes 13 are disposed on the protrusion 131 on the other side to the side contacting the circuit board 5.

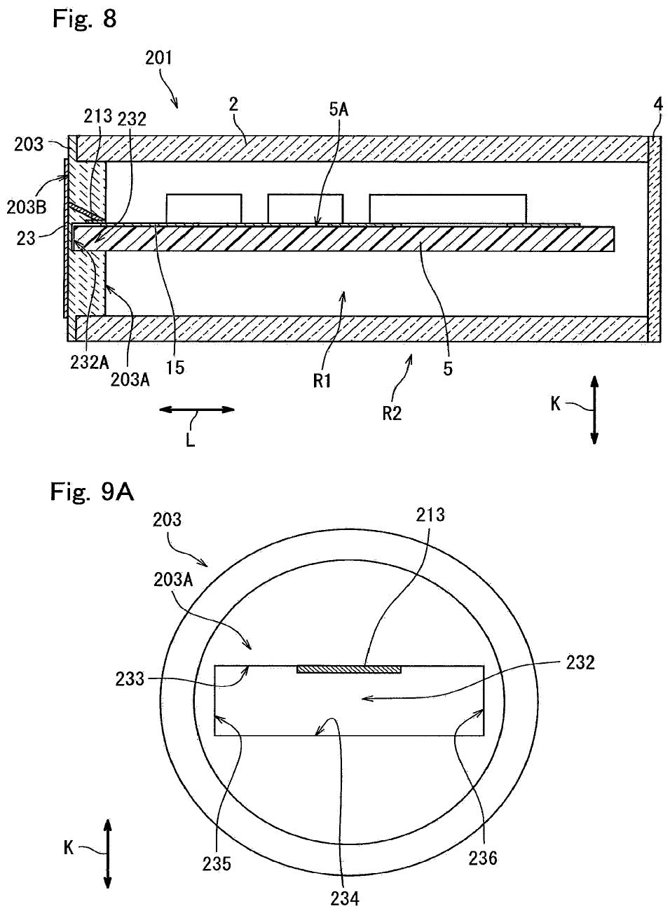

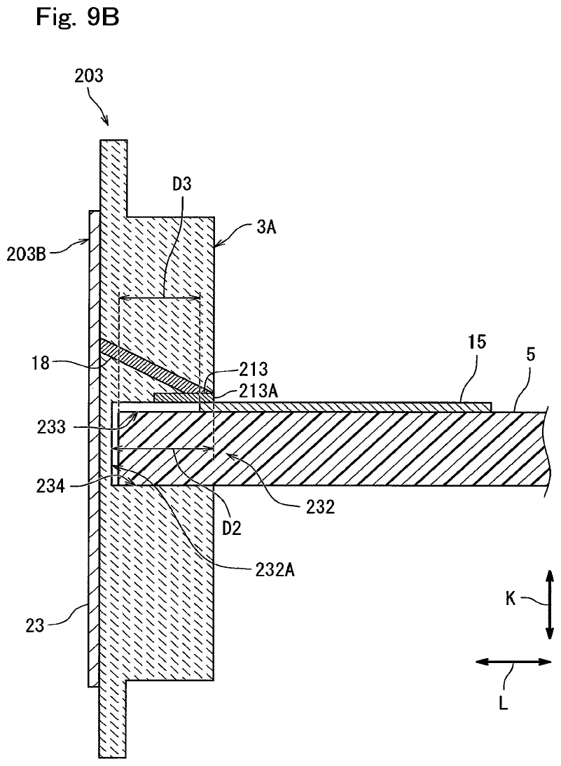

FIG. 8 is a cross-sectional view of the implantable device 201 of the second variation of the exemplary aspect. FIG. 9A is a plane view of the cap 203. FIG. 9B is an enlarged cross-sectional view of the cap 203 and the circuit board 5.

As shown in FIG. 8, the implantable device 201 of the second variation differs from the implantable device 1 of the exemplary aspect described above in that it has a cap 203.

As shown in FIG. 8 and FIG. 9A, the inner surface 203A of the cap 203 has a recess 232. The circuit board 5 is inserted into the recess 232. It is possible to align the circuit board 5 to the cap 203, using recess 232. The end of the circuit board 5 may contact the bottom 232A of the recess 232.

The recess 232 has a rectangular opening. Specifically, the recess 232 is defined by inner walls 233, 234 and inner walls 235, 236. The inner walls 233, 234 face each other and the inner walls 235, 236 face each other. The spacing between the inner walls 233, 234 correspond to the thickness of the circuit board 5, and the inner wall 233 contacts the active surface 5A of the circuit board 5, while the inner wall 234 contacts the reverse side of the circuit board 5. On the other hand, the spacing between the inner walls 235, 236 corresponds to the width of the circuit board 5, and the inner walls 235, 236 contact the side walls of the circuit board 5. According to this configuration, the circuit board 5 can be sandwiched between the inner walls 233, 234, 235, 236, and align the circuit board 5 to the cap 203 and hold it in a fixed manner. In the case that the cap 203 is made of an elastic material, the circuit board 5 can be retained in the recess 232 in a more stable manner.

As shown in FIG. 9B, the depth of the recess 232 from the opening to the bottom 232A is greater than the distance D3 from the end of the circuit board 5 to the board electrode 15. Therefore, at least a portion of the board electrode 15 is placed inside the recess 232, and contacts the inner wall 233.

The inner electrode 213 is positioned on the inner wall 233, and the board electrode 15 is positioned on the part of the circuit board 5 which is disposed in the recess 232. The distance D2 between the bottom 232A of the recess 232 and the tip 213A of the inner electrode 213 distal to the bottom 232A is greater than the distance D3 between the end of the circuit board 5 and the board electrode 15. Therefore, the inner electrode 213 contacts the board electrode 15. Thus, electrical connectivity can be achieved between the inner electrode 213 and the board electrode 15, even when the connection member 17 is not provided.

According to this configuration, by positioning the circuit board 5 in the recess 232, electrical connectivity can be achieved between the cap 203 and the board 5. Therefore, it is possible to omit other components used to achieve an electrical connection, and thus make the implantable device 201 smaller and its structure more simple.

The connection member 17, such as an adhesive, may be provided in the recess 232.

In the second variation, protrusions may be provided in the area surrounding the recess 232. For example, a first protrusion and a second protrusion may be provided on the inner surface 203A on opposing sides of the recess 232, wherein the inner wall 233 is part of the first protrusion, the inner wall 234 is part of the second protrusion and the recess 232 is between the first protrusion and the second protrusion.

In the second variation, an example, in which the inner electrode 213 is disposed on the inner wall 233, is described, but the disclosure is not limited to this configuration. For example, the inner electrode 213 may be disposed on part of the inner surface 203A surrounding the recess 232.

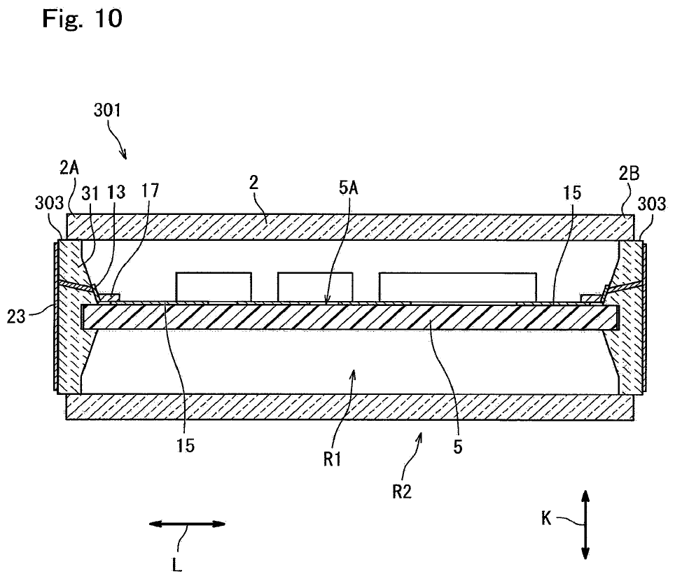

FIG. 10 is a cross-sectional view of the implantable device 301 of the third variation of the exemplary aspect. As shown in FIG. 10, the implantable device 301 of the third variation differs from the implantable device 1 of the exemplary aspect described above in that it has a cap 303.

The implantable device 301 is provided with the body 2 and two caps 303. The body 2 has both ends 2A, 2B open and the two caps 303 close both ends 2A, 2B of the body 2. The outer circumferential surface of the caps 303 contacts the inner surface of the body 2, to seal the body 2. The caps 303 may be caulked to the ends 2A, 2B of the body 2.

The outer diameter of the cap 303 is less than the inner diameter of the body 2. Therefore, the caps 303 can pass through the body 2 from one end to the other. According to this configuration, during the assembly of the implantable device 301, both caps 303 can be attached to the circuit board 5 and the caps 303 can be connected using the connection member 17, and then to insert the integrated structure of the caps 303 with the circuit board 5 into the body 2. Therefore, it is easy to assemble the implantable device 301, even in cases where the implantable device 301 has two caps 303.

Furthermore, it is possible to combine one cap 303 with another cap. Even in this case, by passing the end of the circuit board with the cap 303 attached to it through the body 2, it is possible to assemble the circuit board 5 and both caps before inserting the pre-assembled structure into the body 2.

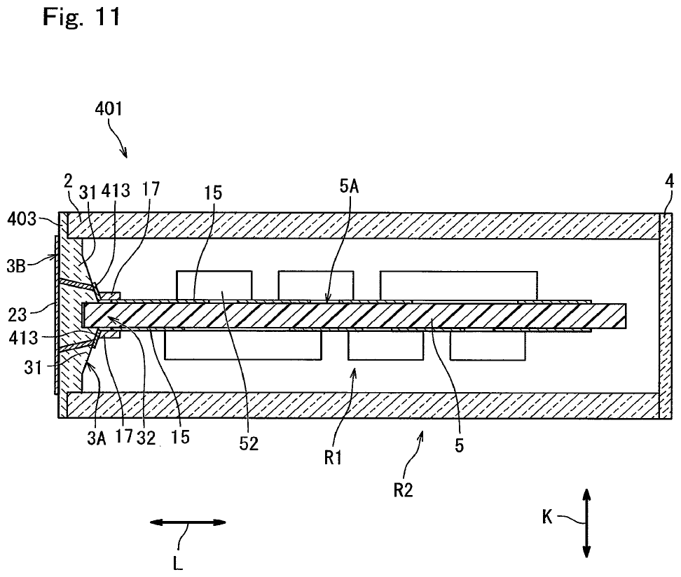

FIG. 11 is a cross-sectional view of the implantable device 401 of the fourth variation of the exemplary aspect. As shown in FIG. 11, the implantable device 401 of the fourth variation differs from the implantable device 1 of the exemplary aspect described above in that it has a cap 403.

On the inner surface 403A of the cap 403, each of the two protrusions 31 have an inner electrode 413. The two inner electrodes 413 are arranged so that they contact opposite sides of the circuit board 5, and are symmetrical about the circuit board 5 in the thickness direction K.

In order to validate the accuracy in estimating the heart rate (HR) and the breathing rate (BR) by the implantable device 1, an experiment comparing the implantable device 1 to conventional ECG electrodes was carried out. All animal procedures were approved by the Institutional Animal Care and Use Committee (IACUC) of NC State University.

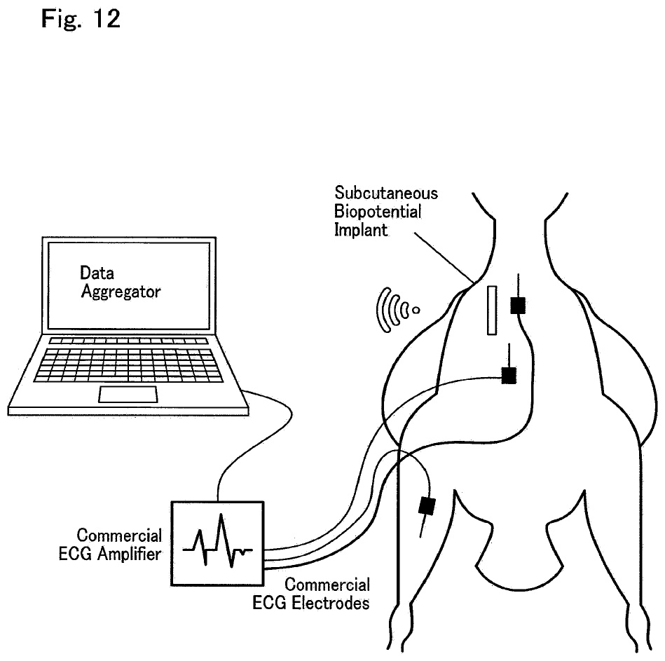

FIG. 12 is a schematic view showing a practical example of the implantable device 1.

As shown in FIG. 12, a proof-of-concept in vivo experiment was performed using a 22-week old White Leghorn chicken (Gallus gallus domesticus) subject. As for the conventional ECG electrodes, needle electrodes connected to a commercial device (for example, Go Direct™ EKG Sensor, Vernier Software & Technology, Beaverton, OR, USA) were used.

During this experiment, local anesthesia was applied to the dorsal side of the chicken and a few feathers were removed to prepare the implantation site for the implantable device 1. Then, the implantable device 1 was subcutaneously injected into the chicken at the nape of the neck between the shoulder blades and a wireless data connection was established between the implantable device 1 and a remote laptop computer. To record ECG signals from both devices simultaneously, three needle electrodes were attached subdermally for the Go Direct™ device. Two needles were inserted adjacent to the implantable device 1 and the third one was inserted in the right leg. The Go Direct™ device was connected to a computer through a USB cable.

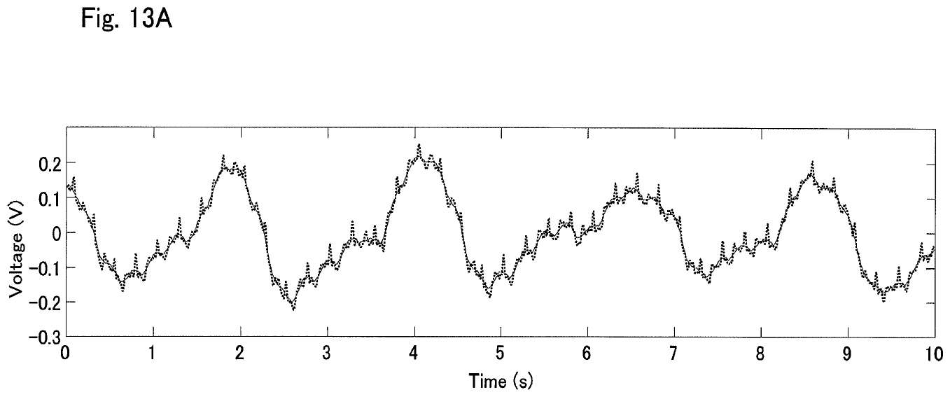

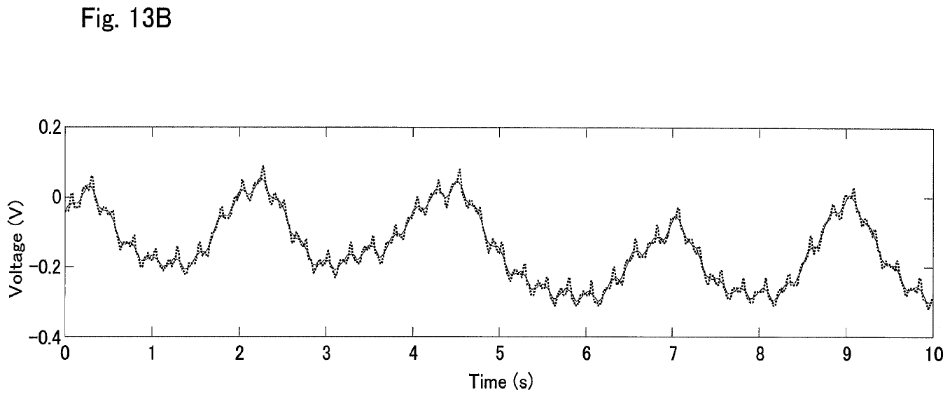

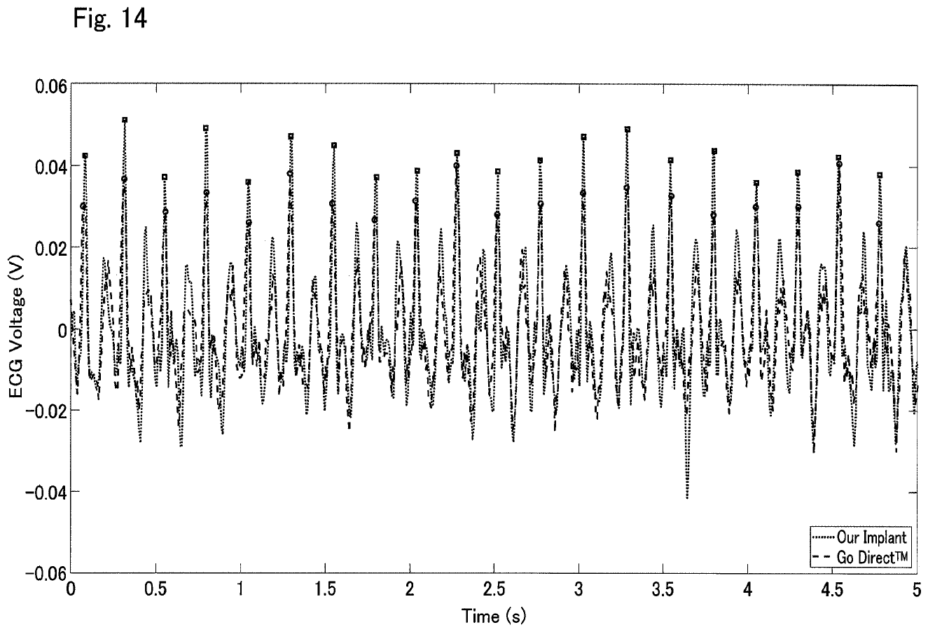

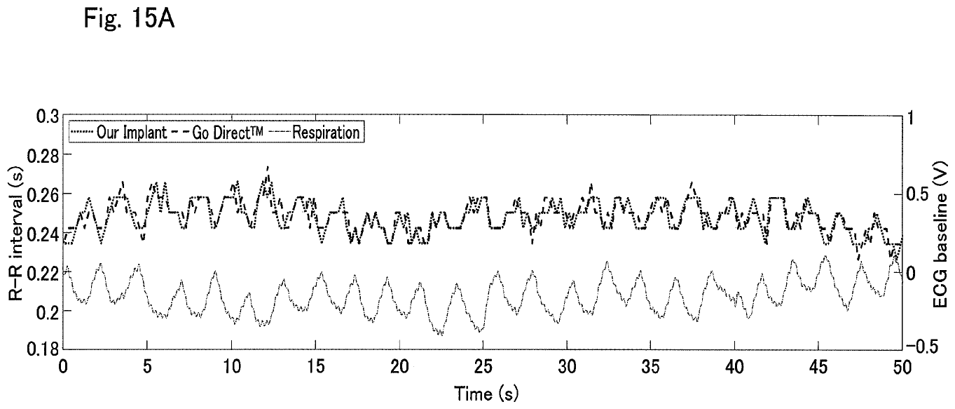

FIG. 13A is a figure showing raw ECG signals (dotted line) modulated by respiratory artifacts recorded by the implantable device 1 using a sampling frequency of 128 Hz. FIG. 13B is a figure showing raw ECG signals (dotted line) modulated by respiratory artifacts recorded by the conventional ECG electrodes using a sampling frequency of 128 Hz. In both FIG. 13A and FIG. 13B, the thin solid line represents the baseline for respiratory modulation. FIG. 14 is a figure showing filtered ECG signals recorded by the implantable device 1 and the conventional ECG electrodes after subtracting the respiratory artifacts. In FIG. 14, the R-peaks of the cardiac cycles recorded by the implantable device 1 are enclosed by a square, while the R-peaks of the cardiac cycles recorded by the conventional ECG electrodes are circled.

A 15-point moving average filter was used to extract the respiratory baselines. As shown in FIG. 14, once the baselines are subtracted from the raw ECG signals, the clean ECG signals were obtained. For both devices, the clean ECG signals coincided with each other.

Peak detection was performed using a library function “findpeaks” in MATLAB™ (for example, Mathworks, Natick, MA, USA) on the filtered ECG signals and the ECG baselines to get the R-R and respiratory intervals, respectively. The filtered ECG signals and the ECG baselines are 3-minutes long.

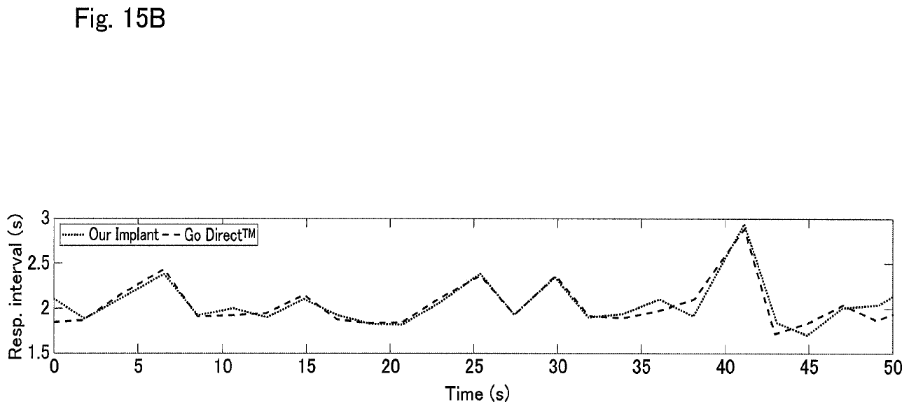

FIG. 15A is a figure showing R-R intervals extracted using the clean ECG signals obtained from both devices. FIG. 15B is a figure showing respiratory intervals calculated from the gap between consecutive peaks in the ECG baseline. In FIG. 15A, the respiratory baseline is also shown on the plot by a dash-dot line to demonstrate the effect of respiratory sinus arrhythmia.

Heart rate in beats per minute (BPM) and breathing rate in breaths per minute (BrPM) were calculated from the peak-to-peak intervals shown in FIG. 15A and FIG. 15B.

TABLE 1

| Quantity (unit) | | Presented Implant | Go Direct ™ |
|---|---|---|---|
| Heart rate (BPM) | Mean | 244.64 | 244.20 |
| | SD | 8.87 | 9.03 |
| Breathing rate (BrPM) | Mean | 28.71 | 28.70 |
| | SD | 2.61 | 2.67 |

As illustrated in Table 1, the mean absolute percentage error and the Pearson correlation coefficient between the heart rate values from the two devices were 1.72% and 0.77, while those between the breathing rate values from the two devices were 4.38% and 0.79, respectively. These rates, as well as the other statistics calculated from these rates, demonstrate the ability of the implantable device 1 to provide accurate physiological information.

In FIG. 15A, a periodic modulation in heartbeat intervals (or heart rate) linked with respiratory artifacts, which is known as respiratory sinus arrhythmia (RSA), was observed. This physiological phenomenon, which causes the R-R interval to shorten during inhalation and lengthen during exhalation, is common in humans (especially neonates) and other vertebrates. In FIG. 15A, the respiratory baseline is shown in the plot in order to show the effect of RSA. To verify the effect of RSA on the R-R intervals, we applied a Discrete Fourier Transform (DFT) to the transient signals of R-R interval values and the respiratory baseline of the ECG waveform collected from the implantable device 1.

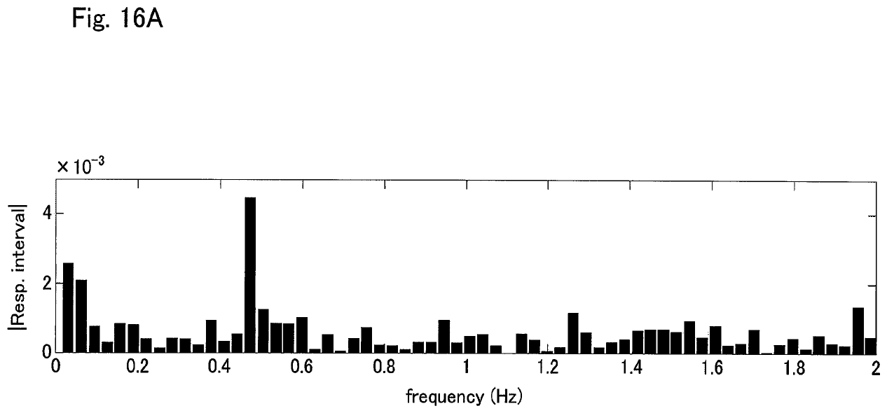

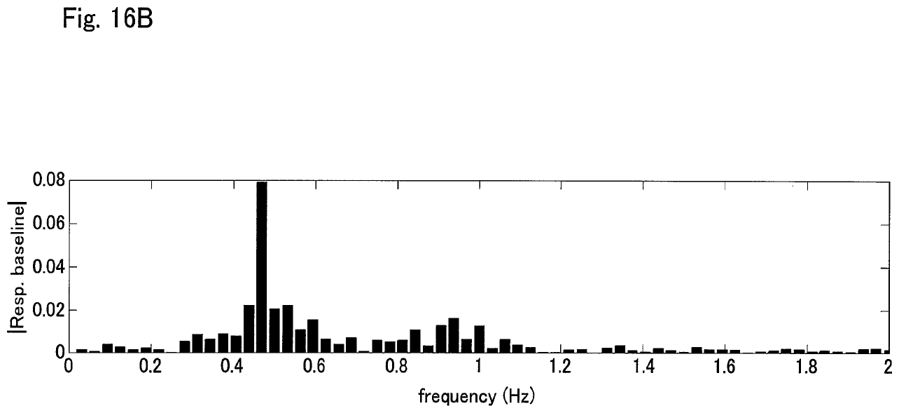

FIG. 16A is a figure showing the magnitude of DFT applied on the R-R interval values from the implantable device 1. FIG. 16B is a figure showing the magnitude of DFT applied on the respiratory baseline.

Both signals showed peaks at the same frequency corresponding to the BR. The synchronized peak at approximately 0.47 Hz (28.2 BrPM) confirms the effect of respiratory sinus arrhythmia. This result reinforces the functionality of the implantable device 1 in capturing useful physiological insight.

The implantable device 1 has been developed as a subcutaneously implantable biopotential sensor system to record continuous ECG for extracting heart rate and breathing rate in animals. Conventionally, the design and manufacturing of such implantable devices had been hindered by the difficulties involved with biopotential electrode interfacing and its integration into miniaturized packaging. In order to facilitate the mass production of biopotential electrodes for the implantable device 1, a multi-material 3D-printing technology is used. In the practical example, it was shown that the implantable device 1 has an accuracy comparable to that of a commercial benchtop ECG system.

It is noted that the present disclosure is sufficiently described in relation to the exemplary aspects and with reference to the appended figures. However, various changes and modifications are obvious to those skilled in the art. The configurations of the aspects and the variations described above may be combined with each other.

The implantable device of the present disclosure is easy to assemble and as such, is useful as an implantable device for animal scientists, veterinarians, and biomedical researchers for monitoring biological information continuously in real-time and with a high resolution.

In general, the description of the aspects disclosed should be considered as being illustrative in all respects and not being restrictive. The scope of the present invention is shown by the claims rather than by the above description, and is intended to include meanings equivalent to the claims and all changes in the scope. While preferred aspects of the invention have been described above, it is to be understood that variations and modifications will be apparent to those skilled in the art without departing from the scope and spirit of the invention.

1. An implantable device, comprising:
a tubular body with at least one open end that defines an inner space;
a cap closing the at least one open end and having an inner surface on one side and an outer surface on the other side, the inner surface facing the inner space; and
a circuit board disposed in the inner space along an axial direction passing from the at least one open end to another end, and having an active surface along the axial direction, a board electrode disposed on the active surface, and a wireless communication circuit configured to communicate biological information obtained via the board electrode,
wherein a protrusion is disposed on the inner surface of the cap and contacts the active surface,
wherein an inner electrode is disposed on the protrusion and is electrically connected to the board electrode,
wherein an outer electrode is disposed on the outer surface of the cap and is electrically connected to the inner electrode,
wherein the implantable device further comprises a connection member that connects the inner electrode and the board electrode,
wherein the protrusion has on one side a first surface contacting the active surface and on another side a second surface facing a direction that intersects the axial direction, and
wherein the inner electrode is disposed on the second surface of the protrusion, and the connection member is electrically conductive.

2. The implantable device according to claim 1, wherein the cap has an outer diameter that is less than an inner diameter of the tubular body.

3. The implantable device according to claim 1,
wherein the at least one open end is a first open end, the cap is a first cap, and the tubular body has a second open end that is opposite the first open end,
wherein the implantable device further comprises a second cap that closes the second open end, and
wherein the first cap and the second cap each have an outer diameter that is less than an inner diameter of the tubular body.

4. The implantable device according to claim 1,
wherein the cap comprises a feed-through conductor that penetrates the cap and that electrically connects the outer electrode and the inner electrode, and
wherein the feed-through conductor is tilted with regards to a thickness direction of the cap.

5. The implantable device according to claim 1,
wherein the protrusion is a first protrusion,
wherein the implantable device further comprises a second protrusion disposed on the inner surface of the cap, and
wherein the circuit board is disposed between the first protrusion and the second protrusion.

6. The implantable device according to claim 5,
wherein a recess is formed between the first protrusion and the second protrusion, and
wherein the circuit board is disposed in the recess.

7. The implantable device according to claim 6, wherein the recess is defined by an inner wall having a first inner wall and a second inner wall facing each other.

8. The implantable device according to claim 7, wherein the circuit board contacts at least one of the first inner wall or the second inner wall.

9. The implantable device according to claim 8, wherein the circuit board contacts both the first inner wall and the second inner wall.

10. The implantable device according to claim 1, wherein the outer electrode has a larger surface area than the inner electrode.

11. The implantable device according to claim 1, wherein the cap comprises a ceramic material and a metal material.

12. The implantable device according to claim 1, wherein the second surface is tilted so that the second surface approaches the active surface.
